(19) United States
(12) Patent Application Publication
(10) Pub. No.: US 2019/0381101 A1
(43) Pub. Date: Dec. 19, 2019

(54) CALRETICULIN-MEDIATED CANCER TREATMENT

(71) Applicant: NANT HOLDINGS IP, LLC, Culver City, CA (US)

(72) Inventor: Patrick SOON-SHIONG, Culver City, CA (US)

(21) Appl. No.: 16/482,184

(22) PCT Filed: Feb. 1, 2018
(86) PCT No.: PCT/US2018/016513

§ 371 Date: Jul. 30, 2019

Related U.S. Application Data
(60) Provisional application No. 62/453,229, filed on Feb. 1, 2017.

(51) Int. Cl. A61K 35/17; C07K 14/735; C07K 16/28

(52) U.S. Cl. CPC A61K 35/17; C07K 14/70535; A61K 31/7068; C07K 16/2863; C07K 16/2827

(57) ABSTRACT

Contemplated compositions and methods take advantage of one or more surface markers on cancer stem cell that are associated with self-protection of tumor cells. Such surface markers are specifically targeted to guide a cell-based cancer treatment, and especially hypoxia resistant NK cells and radiotherapeutics directly to the cancer stem cell. In addition, immune suppression can be counteracted using various inhibitors, while immune response may be further augmented using certain immune stimulatory agents.

This application claims priority to U.S. provisional application Ser. No. 62/453,229, which was filed Feb. 1, 2017, and which is incorporated by reference herein.

### FIELD OF THE INVENTION

The field of the invention is tumor treatments, especially as it relates to compositions and methods to treat a mesenchymal tumor stem cell in a tumor microenvironment.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Cancer stem cells are a subgroup of cells within a tumor and have the ability to self-renew and differentiate to many types of cells in a particular type of tumor to so initiate and sustain the formation and growth of cancer. In many instances, cancer stem cells will cause relapse and metastasis of the tumor, which often also acquires treatment resistance during such process. Several hypotheses have been proposed for the generation of cancer stem cells. Among those, the de-differentiation hypothesis suggests that a mutated cell can be de-differentiated to obtain stem cell-like characteristics. For example, a tumor cell can be transformed to a precursor cell for metastatic cancer cell or cancer stem cell via epithelial-mesenchymal transition (EMT).

EMT is a physiological process during embryogenesis that appears to be reinstated in adult tissues undergoing wound healing and tissue regeneration, or under certain pathological conditions such as fibrosis and cancer. Tumor EMT involves a phenotypic switch that promotes acquisition of a fibroblastoid-like morphology by epithelial tumor cells, that reduces cell polarity and cell-to-cell contacts, and that decreases expression of epithelial markers, including E-cadherin and cytokeratins. On the other hand, epithelial tumor cells undergoing EMT will typically gain expression of mesenchymal-associated proteins, such as fibronectin and vimentin, and will have enhanced cell motility, invasiveness, and metastatic propensity in vivo. Tumor EMT has also been shown to contribute to the acquisition of tumor resistance to chemotherapy, radiation, and certain small-molecule-targeted therapies, thus representing a major mechanism contributing to the progression of carcinomas.

Cancer stem cells are particularly insidious as they tend to develop and maintain their stemness under hypoxic conditions (e.g., Cell Cycle, 2009, 8:20, 3274-3284), and as they often slow down pathways associated with antibody-dependent cell-mediated cytotoxicity (ADCC), apoptosis, and cell division of the tumor cell. In addition, hypoxia also reduces activity of an innate immune reaction (and especially NK activity) to a tumor. Consequently, typical treatments that include chemotherapy and radiation tend to be significantly less effective. Still further, hypoxic conditions also induce up-regulation of TGF-β and IL-8-mediated signaling, which in turn maintains stemness and mesenchymal character of the cancer stem cells, and which tends to attract to and activate myeloid derived suppressor cells (MDSC) in the tumor microenvironment. Thus, cancer stem cells render a tumor not only be more difficult to treat, but also often exhibit self protective mechanisms via suppressor cells.

More recently, the IL-8/IL-8 receptor axis was investigated with respect to the induction and/or maintenance of tumor EMT and its ability to remodel the tumor microenvironment. For example, autocrine loops of IL-8 were suggested to induce and maintain tumor EMT (see e.g., Future Oncol 2012, 8(6): 713-722). Moreover, pharmaceutical intervention on IL-8 signaling was also suggested as a therapeutic approach to halt disease progression driven by IL-8 and other CXCR1/2 ligands (see e.g., Breast Cancer Research 2013, 15:210). Similarly, the IL-8/CXCR1 axis was reported to be associated with cancer stem cell-like properties and to correlate with the clinical prognosis in human pancreatic cancer cases (see e.g., Scientific Reports 2014, 4: 5911), and it was suggested to target pancreatic cancer stem cells by disrupting the IL-8/CXCR1 axis. Interestingly, IL-8 is also a potent chemoattractant for neutrophils and monocytes and has been implicated in directing myeloid derived suppressor cells into the tumor microenvironment (see e.g., Clin Cancer Res 2016, and Vaccines 2016, 4, 22). In yet another example, some myeloid-derived suppressor cells (MDSCs) preferentially infiltrate the tumor and actively induce EMT via transforming growth factor (TGF)-β, epithelial growth factor (EGF) and/or hepatocyte growth factor (HGF)-mediated pathways. However, IL-8 signaling inhibition alone or MDSC inhibition alone has not led to a therapeutically effective path in the treatment of cancer.

Cancer stem cells also express calreticulin on the cell surface, and there are numerous physiological roles reported for calreticulin. While intracellularly (and mostly ER associated) located, calreticulin is involved in chaperoning and protein turnover. On the outside of a cell, calreticulin was reported to act as a signal for macrophage-mediated programmed cell removal (PRCR). The induction of PRCR by ‘eat-me’ signals on tumor cells is countered by ‘don't-eat-me’ signals such as CD47, which binds macrophage signal-regulatory protein alpha to inhibit phagocytosis, and blockade of CD47 on tumor cells has lead to phagocytosis by macrophages (e.g., Proc Natl Acad Sci 2015, 112(7): 2145-2150). Unfortunately, blockade of CD47 has not lead to clinically relevant treatment protocols, possibly due to hypoxic conditions at the tumor microenvironment that up-regulates immune suppression (e.g., due to PD-L1 overexpression).

More recently, it was established that that c-MET (a receptor tyrosine kinase) and its natural ligand HGF (hepatocyte growth factor) are highly expressed in a large number of solid and soft tumors (see e.g., URL: vai.org/met). Moreover, it has been shown that high levels of c-MET can lead to the constitutive activation of the enzyme, as well as rendering cells sensitive to sub-threshold amounts of HGF. Several molecules targeting c-MET have recently been evaluated in early phase clinical trials. Most of them are small kinase inhibitors, while some are biological antagonists and monoclonal antibodies targeting either the ligand or the receptor. For example, a non-ATP competitive c-MET inhibitor, tivantinib, has shown to produce an increased response rate and overall survival when combined with Erlotinib (e.g., J Clin Oncol 2010, 28: LBA7502-LBA7502). Similarly, foretinib, a multikinase inhibitor that targets c-MET and VEGFR2 at nanomolar concentrations was found to stabilize the disease in 55% of the patients treated in a phase I trial (e.g., Clin Cancer Res. 2010, 16(13):3507-16). In addition, MetMAb (OA-5D5), a human, monovalent antagonistic anti-MET antibody (e.g., Cancer Res. 2008; 68(11):4360-8), was able to inhibit glioblastoma U87 and pancreatic BxPC3 and KP4 tumor xenografts. A phase II clinical trial using MetMAb in combination with erlotinib to treat patients with NSCLC resulted in a doubling of patient survival from 6.4 to 12.4 months. However, increased resistance to kinase inhibitors and immunogenicity to antibodies have contributed to difficulties with targeting c-MET.

Even though numerous aspects of tumor cells, and especially stemness of tumor cells are known in the art, none of the insights have led to a therapeutically effective treatment that would help eradicate the tumor. Therefore, there still remains a need for compositions and methods that improve therapy outcome for treatment of a tumor.

### SUMMARY OF THE INVENTION

The inventive subject matter is directed to various compositions and methods in which tumor cells, and especially tumor stem cells, are specifically targeted using surface markers that are characteristic for immune evasion and resistance to traditional treatment methods. Most typically, such features are associated with epithelial to mesenchymal transition (EMT) and include (over-)expression of calreticulin, programmed death ligand 1 (PD-L1) and/or c-MET (tyrosine-protein kinase Met or hepatocyte growth factor receptor), and optionally secreted protein acidic and rich in cysteine (SPARC).

Preferably, thusly targeted cells are subject to immune therapy that includes treatment with cytotoxic immune cells including activated NK (natural killer) cells, genetically modified NK cells or NKT cells that retain cytotoxicity even under hypoxic conditions often found in the tumor microenvironment, which is thought to contribute to or trigger EMT of cancer cells to cancer stem cells. Viewed from a different perspective, it should therefore be appreciated that the inventors contemplate treatment methods and systems that use the very self-protective mechanism of a cancer (stem) cell to effectively target the cancer (stem) cell that is often difficult to treat.

Consequently, in one aspect of the inventive subject matter, the inventors contemplate a method of targeting a cancer stem cell, while in another aspect the inventors contemplate a method of treating cancer, while yet in another aspect the inventors contemplate a method of targeting a cancer cell in a hypoxic environment in which the cancer cell has reduced cell division and/or activity in an apoptotic pathway.

Contemplated methods will typically include a step of providing or co-administering an antibody having binding specificity towards an antigen that is specific to a mesenchymal state of a tumor stem cell and a genetically modified natural killer (NK) cell that expresses a CD16 receptor and that has granulysin and granzyme mediated cytotoxic activity under hypoxic conditions. Thus, cancer (stem) cells will be contacted with the antibody and the genetically modified NK cell to allow antibody-mediated binding of the genetically modified NK cell to the cancer stem cell. Where needed or otherwise desired, types and ratios of antigen expression in the tumor that is specific to a mesenchymal state of a tumor cell or that has undergone EMT in the tumor can be identified and determined before targeting the antigen for the treatment.

In preferred methods, the antigen is calreticulin, PD-L1, and/or c-MET, and the antibody is a human or humanized antibody. Moreover, suitable antibodies also include bi-specific antibodies having binding specificity against at least two of calreticulin, PD-L1, and/or c-MET. With respect to the NK cell it is typically preferred that the NK cell is genetically modified and a NK92 cell. Most preferably, the genetically modified NK cell is modified to express a high affinity variant CD16 (e.g., V158) and/or non-secreted (e.g., endoplasmic restricted) IL-2.

In further contemplated aspects, the tumor stem cell is from a solid tumor, and the step of contacting the cancer stem cell with the antibody and the genetically modified NK cell may be performed while the cancer stem cell is within a tumor microenvironment, and/or under hypoxic conditions. Thus, the cancer stem cell may be contacted with the antibody and the genetically modified NK cell in vivo in a patient, where the cancer stem cell is within a tumor microenvironment.

Additionally, it contemplated that various compounds and compositions may be further administered and/or co-administered to increase an immune reaction against the cancer cell and/or to reduce or eliminate immune suppressive conditions. For example, suitable steps for increasing an immune reaction may include a step of administering to the cancer stem cell or a tumor microenvironment an immune stimulating cytokine (e.g., IL-15, an IL-15 superagonist, and/or an IL-15 superagonist hybrid comprising a chemokine or chemokine portion such as CXCL14, etc.), a chemokine (e.g., CXCL14, etc.) that attracts a T cells and/or NK cells, additional oxygen (e.g., via oxygen hyperbaric treatment), and/or a radiosensitizing drug (e.g., via coupling of the drug to albumin that is transported via SPARC). Moreover, immunogenicity may be further enhanced by administering to the cancer stem cell or tumor microenvironment a CD47 antagonist or a SHPS-1 antagonist, and/or by administering to the cancer stem cell or tumor microenvironment an agent that up-regulates surface expression of calreticulin (e.g., an anthracycline or thapsigargin). Alternatively, or additionally, contemplated methods may also include a step of administering to the cancer stem cell or tumor microenvironment an antibody or its fragment thereof that binds to the antigen and that further comprises an alpha or beta radioisotope.

In other examples for reducing or eliminating immune suppressive conditions, suitable methods may include a further step of administering to the cancer stem cell or tumor microenvironment an agent that down-regulates myeloid derived suppressor cells (e.g., gemcitabine, cis-platinum, cyclophosphamide, etc.), a peptide that down-regulates or kills M2 macrophages (e.g., riptide 182 peptide or antibody against B7-H4, etc.), an IL8/CXCR1/CXCR2 signaling pathway inhibitor (e.g., IL-8 antibody, an IL-8 antagonist, a CXCR1 inhibitor, and/or a CXCR2 inhibitor, etc.).

In further contemplated aspects, the inventors therefore also contemplate the use of (i) an antibody having binding specificity towards an antigen that is specific to a mesenchymal state of a tumor stem cell and (ii) a genetically modified natural killer (NK) cell that expresses a CD16 receptor and that has granulysin and granzyme mediated cytotoxic activity under hypoxic conditions to target a cancer stem cell. Further contemplated uses include a use of (i) an antibody having binding specificity towards an antigen that is specific to a mesenchymal state of a tumor stem cell and (ii) a genetically modified natural killer (NK) cell that expresses a CD16 receptor and that has granulysin and granzyme mediated cytotoxic activity under hypoxic conditions to treat a cancer, and the use of (i) an antibody having binding specificity towards an antigen that is specific to a mesenchymal state of a tumor stem cell and (ii) a genetically modified natural killer (NK) cell that expresses a CD16 receptor and that has granulysin and granzyme mediated cytotoxic activity under hypoxic conditions to target a cancer cell in a hypoxic environment in which the cancer cell has reduced cell division and/or activity in an apoptotic pathway.

Consequently, in yet another aspect of the inventive subject matter, the inventors also contemplate a genetically modified NK cell that expresses a CD16 receptor and that has granulysin and granzyme mediated cytotoxic activity under hypoxic conditions. Further, the genetically modified NK cell includes an antibody bound to the CD16 receptor, where the antibody has binding specificity towards an antigen that is specific to a mesenchymal state of a tumor cell. Viewed from a different perspective, the inventors also contemplate pharmaceutical compositions or kits that include (i) an antibody having binding specificity towards an antigen that is specific to a mesenchymal state of a tumor stem cell, and (ii) a genetically modified NK cell that expresses a CD16 receptor and that has granulysin and granzyme mediated cytotoxic activity under hypoxic conditions.

Various objects, features, aspects and advantages of the inventive subject matter will become more apparent from the following detailed description of preferred embodiments.

### DETAILED DESCRIPTION

In solid tumors, rapid growth of tumor cells often exceeds the speed of vascularization of the neoplasm and will cause hypoxic or nutrition-depleted conditions in the solid tumor. Hypoxic conditions of the tumor provide resistance to many cancer treatments including chemotherapy and immune therapy as it reduces or abrogates apoptotic pathway activity, cell division, anaerobic metabolism, and in some cases even autophagy. In addition, the hypoxic tumor microenvironment also often reduces or blocks cytotoxicity of immune competent cells (e.g., especially NK cells) and further promotes TGF-β and IL-8 secretion, leading to attraction and activation of various suppressor cells. More importantly, hypoxia induces epithelial-mesenchymal transition (EMT) of tumor cells to regain stemness such that so-generated mesenchymal tumor cell can be resistant to many cancer drugs and also evade for metastasis.

As used herein, the term “tumor” refers to, and is interchangeably used with one or more cancer cells, cancer tissues, malignant tumor cells, or malignant tumor tissue, that can be placed or found in one or more anatomical locations in a human body. As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M. As used herein, the term “provide” or “providing” refers to and includes any acts of manufacturing, generating, placing, enabling to use, or making ready to use.

The inventors discovered that cancer (stem) cells can be exquisitely targeted by using molecular markers that are ordinarily associated with the development and maintenance of mesenchymal cancer (stem) cells. The inventors further found that these markers can be further used to actively recruit cytotoxic immune cells (e.g., NK cells, NKT cells, etc.) such that the cytotoxic immune cells selectively induce cytotoxic immune response against the mesenchymal cancer (stem) cells. Thus, in an especially preferred aspect of the inventive subject matter, the inventors contemplate targeting of a cancer stem cell or a cancer cell in a hypoxic environment in which the cancer cell has reduced cell division and/or activity in an apoptotic pathway by a binding molecule to one or more of mesenchymal cancer (stem) cell molecular markers and a cytotoxic immune cell that can attack the mesenchymal cancer (stem) cell by recognizing the binding molecule via CD16 molecule expressed on the surface of the cytotoxic immune cell.

### Molecular Markers

Any suitable molecular markers of mesenchymal cancer (stem) cell that can be recognized by a binding molecule are contemplated. Preferably, the molecular marker is a membrane-bound or membrane-anchored protein, of which at least a portion of the protein is exposed on the surface of the mesenchymal cancer (stem) cell. However, it is also contemplated that the molecular marker can be an intracellular protein, extracellular protein (e.g., extracellular matrix protein or extracellular matrix-binding protein, etc.), or a membrane bound lipid antigen. Furthermore, it is preferred that the molecular marker has a specific or preferential expression in the mesenchymal cancer (stem) cell such that, for example, the expression level of the marker is increased in the mesenchymal cancer (stem) cell compared to other tumor cells in the same tumor (or a similar tumor) at least 20%, at least 30%, at least 50%, at least 70%, or at least 100%.

Among other suitable markers for use herein (e.g., those associated with self-protection of a cancer cell), especially contemplated markers include PD-L1, calreticulin, and c-MET. PD-L1 (also known as CD274) an immune inhibitory receptor ligand that is expressed by hematopoietic and non-hematopoietic cells, such as T cells and B cells and various types of tumor cells. The encoded protein is a type I transmembrane protein with immunoglobulin V-like and C-like domains. Interaction of this ligand with its receptor inhibits T-cell activation and cytokine production. In tumor microenvironments, this interaction provides an immune escape for tumor cells through cytotoxic T-cell inactivation. Expression of this gene in tumor cells is considered to be prognostic in many types of human malignancies, including colon cancer and renal cell carcinoma. Alternative splicing results in multiple transcript variants and all variant forms are deemed suitable for use herein, especially human forms. For example, contemplated PD-L1 protein sequences and isoforms include those known from NCBI Reference sequences NP_054862.1 (isoform 1a), NP_001300958.1 (isoform 1c), and NP_001254635.1 (isoform 1b), encoded by NCBI Genomic Reference sequence NC_000009.12 or NC_018920.2. Of course, it should be appreciated that the exact sequence may vary from tumor cell to tumor cell and among different tumors and/or patients. Thus contemplated PD-L1 sequences will include those with at least about 30%, 35%, 40%, 45% or 50%, preferably at least about 55%, 60%, 65% or 70%, and more preferably at least about 75%, 80%, 85%, 90%, 91%, 92%, 93% or 94% and most preferably at least about 95%, 97%, 98%, 99% or more homology to the sequences as described above.

Calreticulin (also known as CRP55) is highly conserved among species and is known as a multifunctional protein acting as a major Ca²⁺-binding/storage protein in the lumen of the endoplasmic reticulum, but is also found in the nucleus, suggesting that it may have a role in transcription regulation. Calreticulin binds to the synthetic peptide KLGFFKR, which is almost identical to an amino acid sequence in the DNA-binding domain of the superfamily of nuclear receptors. The amino terminus of calreticulin interacts with the DNA-binding domain of the glucocorticoid receptor and prevents the receptor from binding to its specific glucocorticoid response element. Calreticulin can inhibit the binding of the androgen receptor to its hormone-responsive DNA element and can inhibit both androgen receptor and retinoic acid receptor transcriptional activities in vivo, as well as retinoic acid-induced neuronal differentiation. Thus, calreticulin can act as an important modulator of the regulation of gene transcription by nuclear hormone receptors. In addition, while calreticulin, in general, is commonly expressed in endoplasmic reticulum (ER) with an ER retention signal KDEL, cell surface expression of calreticulin is increased in tumor cells, especially in the cancer stem cells, under a hypoxic condition in the tumor microenvironment. Thus, surface expressed calreticulin in the tumor can be used as a marker for the mesenchymal cancer (stem) cells. For example, contemplated calreticulin protein sequences include those known from NCBI Reference sequences NP_004334.1, encoded by NCBI Genomic Reference sequence NG_029662.1. As noted above, it should be appreciated that the exact sequence may vary from tumor cell to tumor cell and among different tumors and/or patients. Thus contemplated calreticulin sequences will include those with at least about 30%, 35%, 40%, 45% or 50%, preferably at least about 55%, 60%, 65% or 70%, and more preferably at least about 75%, 80%, 85%, 90%, 91%, 92%, 93% or 94% and most preferably at least about 95%, 97%, 98%, 99% or more homology to the sequences as described above.

c-Met is a member of the receptor tyrosine kinase family of proteins and the product of the proto-oncogene MET. The encoded preproprotein is proteolytically processed to generate alpha and beta subunits that are linked via disulfide bonds to form a mature receptor. Binding of its ligand, hepatocyte growth factor, induces dimerization and activation of the receptor, which plays a role in cellular survival, embryogenesis, and cellular migration and invasion. Mutations in this gene are associated with papillary renal cell carcinoma, hepatocellular carcinoma, and various head and neck cancers. Amplification and overexpression of this gene are associated with multiple human cancers as noted above. Alternative splicing results in multiple transcript variants and all variant forms are deemed suitable for use herein, especially human forms. For example, contemplated c-Met protein sequences and isoforms include those known from NCBI Reference sequences NP_001120972.1 (isoform a), NP_000236.2 (isoform b), NP_001311330.1 (isoform c), and NP_001311331.1 (isoform d), encoded by NCBI Genomic Reference sequence NG_008996.1. Of course, it should be appreciated that the exact sequence may vary from tumor cell to tumor cell and among different tumors and/or patients. Thus contemplated cMet sequences will include those with at least about 30%, 35%, 40%, 45% or 50%, preferably at least about 55%, 60%, 65% or 70%, and more preferably at least about 75%, 80%, 85%, 90%, 91%, 92%, 93% or 94% and most preferably at least about 95%, 97%, 98%, 99% or more homology to the sequences as described above.

Optionally, the inventors contemplate that SPARC (also known as osteonectin) can be used as marker for the mesenchymal cancer (stem) cells. SPARC is a cysteine-rich acidic matrix-associated protein, typically required for collagen in bone to become calcified, but also involved in extracellular matrix synthesis and promotion of changes to cell shape. SPARC has also been associated with tumor suppression and has in some cases been correlated with metastasis based on changes to cell shape which can promote tumor cell invasion. Three transcript variants encoding different isoforms have been found for the gene encoding SPARC, and all isoforms are deemed suitable for use herein. Moreover, human forms of SPARC are especially preferred. For example, contemplated SPARC protein sequences and isoforms include those known from NCBI Reference sequences NP_003109.1 (isoform 1), NP_001296372.1 (isoform 2), and NP_001296373.1 (isoform 3), encoded by NCBI Genomic Reference sequence NG_042174.1. Of course, it should be appreciated that the exact sequence may vary from tumor cell to tumor cell and among different tumors and/or patients. Thus contemplated SPARC sequences will include those with at least about 30%, 35%, 40%, 45% or 50%, preferably at least about 55%, 60%, 65% or 70%, and more preferably at least about 75%, 80%, 85%, 90%, 91%, 92%, 93% or 94% and most preferably at least about 95%, 97%, 98%, 99% or more homology to the sequences as described above.

### Binding Molecules

Targeting the markers (as antigens) can be performed in numerous manners, and all known binding molecules that specifically bind to the markers contemplated herein are deemed suitable. However, especially preferred targeting molecules include antibodies and recombinant proteins having an Fc domain and a binding domain coupled to each other. For example, suitable antibodies include IgG, and especially human or humanized antibodies. There are numerous commercial sources for antibodies binding the targets presented herein. For example, monoclonal PD-L1 antibodies are available from Abcam as ab205921 or Ab213524, while calreticulin antibodies are available from Abcam as Ab2907 or Ab22683. Antibodies against c-Met are commercially available from Abcam as Ab51067 or Ab59884. On the other hand, synthetic binders may include an IgG Fc portion that is coupled to a peptide with high affinity to the target (e.g., as identified from an antibody, phage panning, or RNA display). Where desired, the Fc portion may also be modified or optimized for binding to CD16 of a cytotoxic immune cell (e.g., NK cells) as described in more detail further below.

For other example, the targeting molecule can be a hybrid molecule with an Fc domain coupled with one or more fragments of antibodies (e.g., scFv, Fab, F(ab′)₂, etc.). In some embodiments, the hybrid molecule is a bi-specific antibody having an Fc domain coupled with two different Fab arms (e.g., one Fab specific to calreticulin, another Fab specific to PD-L1, etc.) such that the hybrid molecule can concurrently recognize two markers on a mesenchymal tumor cell or two markers on two mesenchymal tumor cells. In such embodiments, heavy chains of the Fc domain may be derived from a single antibody (e.g., a monoclonal calreticulin antibody, etc.) or two different antibodies (e.g., one heavy chain is derived from a monoclonal calreticulin antibody and another heavy chain is derived from a monoclonal PD-L1 antibody, etc.). In both scenarios, it is preferred that the binding affinity of Fc portion of the hybrid molecule to CD16 of the cytotoxic immune cell is not substantially less than the binding affinity of Fc portion of other commercially available monoclonal antibody to CD16.

The inventors contemplate that the targeting molecule can be a targeted ALT-803-based scaffold platform (TxM, Altor Bioscience, 2810 N. Commerce Pwky, Miramar, Fla., 33025) that is coupled with one or more target recognition domains binding at least a portion of PD-L1, calreticulin, and/or c-MET. For example, the targeting molecule can be a TxM that includes two scFvs binding to PD-L1, one scFv binding to calreticulin, and one scFv binding to c-MET, respectively. For other example, the targeting molecule can be a TxM that includes three scFvs binding to PD-L1 and one scFv binding to calreticulin. In some embodiments, the targeting molecule can be coupled with one or more target recognition domain that binds to tumor antigen(s) other than PD-L1, calreticulin, and/or c-MET. For example, a scFv molecule binding to a tumor neoepitope can be generated by first identifying the nucleic acid sequence of V_{H} and V_{L} specific to the tumor neoepitope. In some embodiments, a nucleic acid sequence of V_{H} and V_{L} can be identified from a monoclonal antibody sequence database with known specificity and binding affinity to the tumor epitope. Alternatively, the nucleic acid sequence of V_{H} and V_{L} can be identified via an in silico analysis of candidate sequences (e.g., via IgBLAST sequence analysis tool, etc.). In other embodiments, the nucleic acid sequence of V_{H} and V_{L} can be identified via a mass screening of peptides having various affinities to the tumor neoepitope, tumor associated antigen, or self-lipid via any suitable in vitro assays (e.g., flow cytometry, SPR assay, a kinetic exclusion assay, etc.). In such example, as one targeting molecule binds to both tumor neoepitope (e.g., preferably patient-specific and tumor-specific) and marker antigen(s) of mesenchymal tumor cell, it is contemplated that the specificity of targeting the mesenchymal tumor cell over other types of cells in the tumor or tumor microenvironment can be substantially increased.

Alternatively, where the targeting molecule is an antibody, a TxM or its derivative, it is contemplated that the targeting molecule is coupled to one or more other functional moieties (e.g., radioisotope, cytokine, chemokine, chemotherapeutic drug, etc.) via a linker such that the functional moieties can specifically target and act on tumor microenvironment. For example, the targeting molecule may be coupled with one or more immune-stimulatory molecules (e.g., CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and LFA3, etc.), immune stimulatory cytokines (e.g., IL-2, IL-12, IL-15, IL-15 super agonist (ALT803), IL-21, IPS1, and LMP, etc.), and/or checkpoint inhibitors (e.g., antibodies or binding molecules to CTLA-4 (especially for CD8⁺ cells), PD-1 (especially for CD4⁺ cells), TIM1 receptor, 2B4, and CD160, etc.). Preferably, the linker is a cleavable linker in the acidic environment (e.g., thimaleamic acid linker, acid-cleavable hydrazine, etc.) such that the functional moieties can be released in the acidic tumor microenvironment, where the immune cell activity is substantially reduced.

For other example, the targeting molecule can be coupled with chemotherapeutic drugs or radioisotopes such that the targeting molecule can also be employed as local delivery agents for chemotherapeutic drugs, and more preferably as local delivery agents for site-specific radioisotope treatment using therapeutic alpha and/or beta emitters. Suitable alpha emitters include astatine-211 (²¹¹At, 7.2 h), bismuth-212 (²¹²Bi, 1 h), bismuth-213 (²¹³Bi, 45.6 min), radium-223 (²²³Ra, 11.4 d), actinium-225 (²²⁵Ac, 10.0 d) and thorium-227 (²²⁷Th, 18.7 d), while suitable beta emitters include tungsten-188 (¹⁸⁸W, 69.4 d) and strontium-90 (⁹⁰Sr, 28.8 y). There are numerous methods for incorporation of radioisotopes into antibodies known in the art (see e.g., Acta Oncol. 1993; 32(7-8): 831-9), and all of these are deemed suitable for use herein.

While the molecular markers expressing on the mesenchymal tumor cells can be readily recognized using binding molecules (e.g., antibodies, TxM derived molecule, etc.) described above, targeting mesenchymal tumor cells using a molecular marker that is an intracellular protein or a secreted protein (e.g., extracellular matrix protein) may not be an optimal target as it is generally not associated with the cell membrane. For example, while SPARC can be a reliable marker for targeting mesenchymal tumor cell, SPARC is generally present intracellularly and then secreted to be bound to extracellular matrix at or near the tumor microenvironment. In such example, the inventors contemplate that the binding molecule can be intracellular antibodies (e.g., intrabodies) that are produced in the mesenchymal tumor cell and bind the intracellular marker protein (e.g., SPARC) within the same mesenchymal cell. Preferably, the intracellular antibody can be an scFv fragment specific to SPARC, which is specifically engineered for cytosolic stability, and the recombinant nucleic acid encoding such intracellular antibody can be introduced to the mesenchymal tumor cell via a recombinant virus (e.g., adenovirus, etc.). Alternatively, the scFv specific to SPARC can be internalized to the cell cytoplasm using a carrier-mediated endocytosis (e.g., using nanoparticle, dendrimer, or liposome, etc.).

In some embodiments, the intrabodies specific to SPARC are associated with a lysosomal targeting signal, for example, CD1b leader peptide, transmembrane domain of LAMP, cytoplasmic tail of LAMP (or C-terminus domain of LAMP), or a nucleotide sequence encoding a motif Tyr-X-X-hydrophobic residue. Without wishing to be bound to specific theory, the inventors contemplate that binding of intrabodies to SPARC in the cell cytoplasm would trigger targeting of the SPARC-intrabody complex to the lysosome, where the SPARC peptide fragment can be loaded on the MHC molecule to be presented on the cell surface.

Alternatively, extracellular SPARC (e.g., secreted and/or bound to extracellular matrix) can be targeted with a binding molecule (e.g., antibody binding to SPARC, TxM having an scFv fragment binding to SPARC), which is preferably coupled with one or more functional moieties (e.g., radioisotope, cytokine, chemokine, chemotherapeutic drug, etc.). In this embodiment, the presence and detection of SPARC using binding molecules in the extracellular matrix near the mesenchymal tumor cells in the tumor allows localized and targeted application and/or release of therapeutic drugs and/or immune-stimulatory cytokines or chemokines.

### Cytotoxic Immune Cells

While any suitable immune cells (either naïve or genetically engineered) that can be activated upon recognizing the binding molecule-marker complex are contemplate, it is preferred that the immune cells express CD16 that binds to Fc domain, where the binding molecules includes Fc domain (of antibodies or TxM). Thus, most preferred immune cells may include CD16+ NK cells and/or NKT cells.

With respect to NK cells, NK cells can be readily identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Of course, it should be noted that suitable host cells, and particularly NK cells are either obtained from the patient diagnosed with the tumor, or are obtained from an already established cell line as further detailed below.

It is preferred that autologous NK cells from the patient as well as NK cells grown from precursor cells of the same patient are used to treat the patient to reduce any allograft rejection in the patient. In such case, it is preferred that patient's NK cells are isolated from the patient's blood using CD16 and/or other molecular markers of NK cells (e.g., CD56, etc.), and optionally expanded/activated ex vivo using, for example, a combination of IL-2 and α-CD3 antibody, or in presence of accessory cells (e.g., monocytes, B-lymphoblastoid cells, K562 cells, etc.).

With respect to NKT cells, NKT cells represent a heterogeneous cell population that can be grouped into three categories based on presence of several molecular markers (e.g., Vα24, etc.) and/or their reactivity to a ligand (e.g., CD1d-restricted, reactivity to α-galactosylceramide (α-GalCer), etc.). In one embodiment, isolation of human type I NKT cells, which typically express Vα24-Jα18 type T cell receptor, can be performed using an antibody against Vα24 or an antibody against Vα24-Jα18. In other embodiments, isolation of human type I and type II NKT cells, which are typically CD1d-restricted cells, can be performed using a portion of CD1d molecule (preferably the portion that are responsible for a high affinity to NKT T cell receptor), a portion of CD1d molecule coupled with a lipid antigen (e.g., any lipid antigens that are generated from a foreign organism, nutritional substances, or self-lipids generated from the patient that can bind to CD1d, etc.), or a portion of CD1d molecule coupled with a peptide (e.g., p99, etc.). Once isolated, the population of isolated and enriched NKT cells can be further increased via ex vivo expansion of the NKT cells. The ex vivo expansion of NKT cells can be performed in any suitable method with any suitable materials that can expand NKT cells at least 10 times, preferably at least 100 times in 7-21 days. For example, isolated and enriched NKT cells can be placed in a cell culture media (e.g., AIMV® medium, RPMI1640® etc.) that includes one or more activating conditions. The activating conditions may include addition of any molecules that can stimulate NKT growth, induce cell division of NKT, and/or stimulate cytokine release from NKT that can further expand NKT cells. Thus the activating molecules include one or more cytokines (e.g., IL-2, IL-5, IL-7, IL-8, IL-12, IL-12, IL-15, IL-18, and IL-21, preferably human recombinant IL-2, IL-5, IL-7, IL-8, IL-12, IL-12, IL-15, IL-18, and IL-21, etc.) in any desirable concentration (e.g., at least 10 U/ml, at least 50 U/ml, at least 100 U/ml), T cell receptor antibodies (e.g., anti-CD2, anti-CD3, anti-CD28, α-TCR-Vα24+ antibodies, preferably immobilized on beads, etc.), a glycolipid (e.g., α-GlcCer, β-ManCer, GD3, etc.), a glycolipid coupled with CD1 (e.g., CD1d, etc.), etc.

However, it is also contemplated that the NK cells and/or NKT cells may also be heterologous NK cells and/or NKT cells. For example, preferred NK cells may include immortalized NK cells (typically irradiated prior to administration) , and such immortalized NK cells include NK92 cells that may be genetically engineered to achieve one or more specific purpose. For example, NK cell is a NK92 cell that has a recombinant high affinity variant of CD16 (e.g., V158 variant). In addition, it is also preferred that the NK92 cell is further genetically modified to express IL-2 in the endoplasmic reticulum such that the cytotoxicity of NK cell remains active under hypoxic conditions (e.g., tumor microenvironment). One of the preferred types of NK cells includes commercially available haNK cells from NantKwest (9920 Jefferson Blvd. Culver City, Calif. 90232). Where desired, such NK cells may be further genetically modified with a recombinant nucleic acid that includes a hypoxia sensitive promotor (e.g., hypoxia response element).

In detail, the NK cell can be a NK92 derivative and is preferably genetically modified to have a reduced or abolished expression of at least one killer cell immunoglobulin-like receptor (KIR), which will render such cells constitutively activated (via lack of or reduced inhibition). Therefore, suitable modified cells may have one or more modified killer cell immunoglobulin-like receptors that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest (see URL www.nantkwest.com) as aNK cells (‘activated natural killer cells).

The genetically engineered NK cell may also be an NK92 derivative that is modified to express the high-affinity Fcγ receptor (CD16) as noted above. Sequences for high-affinity variants of the Fcγ receptor are well known in the art, and all manners of generating and expression are deemed suitable for use herein. Expression of such receptor is believed to allow specific targeting of tumor cells using antibodies that are specific to a patient's tumor cells (e.g., neoepitopes, etc.), a particular tumor type (e.g., her2neu, PSA, PSMA, etc.), or that are associated with cancer (e.g., CEA-CAM, etc.). Advantageously, such antibodies are commercially available and can be used in conjunction with the cells (e.g., bound to the Fcγ receptor). Alternatively, such cells may also be commercially obtained from NantKwest as haNK cells (‘high-affinity natural killer cells). Most notably, such NK cells may be further modified to express non-secreted IL-2, which advantageously renders such NK cells active in a hypoxic microenvironment.

In some embodiments, the genetically engineered NK cell may also be genetically engineered to express a chimeric T-cell receptor. In especially preferred aspects, the chimeric T-cell receptor will have a scFv portion or other ectodomain with binding specificity against a tumor associated antigen, a tumor specific antigen, and a cancer neoepitope. As noted before, there are numerous manners of genetically engineering an NK cell to express such chimeric T-cell receptor, and all manners are deemed suitable for use herein. Alternatively, such cells may also be commercially obtained from NantKwest as taNK cells (‘target-activated natural killer cells’). Where the cells are engineered to have affinity towards a cancer associated antigen or antibody with specificity towards a cancer associated antigen, it is contemplated that all known cancer associated antigens are considered appropriate for use. For example, cancer associated antigens include CEA, MUC-1, CYPB1, etc. Likewise, where the cells are engineered to have affinity towards a cancer specific antigen or antibody with specificity towards a cancer specific antigen, it is contemplated that all known cancer specific antigens are considered appropriate for use. For example, cancer specific antigens include PSA, Her-2, PSA, brachyury, etc.

In other embodiments, the genetically engineered NKT cell may be genetically modified for specific targeting to tumor cells and/or increasing the effect of NKT cell immune in suppressing the activity of myeloid-derived suppressor cells. For example, the inventors contemplate that NKT cells can be genetically modified to specifically recognize a tumor specific or tumor associated antigen, a neoepitope, and/or a self-lipid expressed by the tumor cell by introducing a recombinant protein to the NKT cells. In one embodiment, the NKT cells can be genetically engineered to express a chimeric antigenic receptor (CAR) that includes a specific binding domain (e.g. scFv portion, etc.) to specifically recognize a tumor specific or tumor associated antigen, a neoepitope, and/or a self-lipid expressed by the tumor cell, a transmembrane domain and an intracellular activation domain that may vary depending on the cell type (e.g., a plurality of ITAM motif-including activation domain, etc.).

In addition, it is contemplated that the NK or NKT cells (or other immune competent cells) may be genetically modified to express one or more proteins that support, activate, or provide a desired function to the transfected cells. For example, the NK or NKT cells (or other immune competent cells) may express at least a portion of IL2RA, optionally together with one or more of IL2RB and IL2RG to provide an extra avenue for NK cell activation and to so enhance a more robust immune response. For example, genetically engineered NK cells will most preferably be activated NK cells, high-affinity NK cells, or target activated NK cells. Preferred IL2RA include full length or high-affinity variants of IL2RA. In addition, it is contemplated that the genetically engineered NK cells may also express one or more cytokines, and especially IL-12. Thus, it should be appreciated that the so prepared NK cells may outcompete the hosts T-cells for IL-2. Moreover, contemplated NK or other host cells may also express IL-15 or an IL-15 superagonist (e.g., ALT-803) to so provide increased activation. Finally, where desired, the NK or other host cells may express one or more immune checkpoint inhibitors to further enhance or stimulate the host immune response.

In yet another example, the inventors contemplate transfection of genetically engineered NK or NKT cells (or other immune competent cells) to express one or more co-stimulatory molecules to so enhance an immune response. Once more, the genetically engineered NK cells will most preferably be activated NK cells, high-affinity NK cells, or target activated NK cells. Preferred co-stimulatory molecules can be B7.1 (CD80), ICAM-1 (CD54), ICOS-L, and/or LFA-3 (CD58). In another example, preferred co-stimulatory molecules can be 4-1BBL, CD30L, CD40, CD40L, CD48, CD70, CD112, CD155, GITRL, OX40L, and/or TL1A, optionally in combination with any one of B7.1 (CD80), ICAM-1 (CD54), ICOS-L, and/or LFA-3 (CD58).

Where desired, modified NK cells may also present at least a portion of CXCL12, more preferably a full length CXCL12, and/or that the NK cells are genetically modified to reduce or even entirely silence expression of the CXCR4. By presentation of at least a portion of CXCL12 on the surface of the NK cells and/or removal of the CXCR4, it is believed that the so modified cells will be less subject to recognition and allograft rejection by the host and will have a reduced propensity to aggregate, while still retaining killing activity via NK cell-specific pathways.

### Administration of Binding Molecules and Cytotoxic Immune Cells

As cancer cells in a hypoxic environment will often undergo EMT and develop into cancer stem cells, and as cancer stem cells and other cancer cells tend to express on their surface one or more surface markers associated with self-protection, it should be appreciated in view of the above that cancer cells, and especially cancer stem cells may be targeted with antibodies that ‘tag’ a cancer cell or cancer stem cell, which will then serve as an activation signal for NK cells, and particularly NK cells that are not inhibited by a hypoxic microenvironment. Therefore, and viewed form a different perspective, it should be recognized that cancer stem cells that are ordinarily difficult to treat (e.g., due to reduced activity in metabolism, reduced activity in apoptotic pathways, and reduced cell division) can now be specifically targeted by the very mechanism that these cells employ for self-protection.

Thus, in one embodiment, the mesenchymal tumor cell can be targeted by contacting the mesenchymal tumor cell expressing one or more molecular markers (e.g., calreticulin, PD-L1, and c-MET) with a binding molecule (e.g., antibody, scFv fragment, TxM scaffold coupled with scFv) to the molecular markers such that the molecular marker and the binding molecule can form a protein complex. Then, the mesenchymal tumor cell having the protein complex on its surface is further contacted with a cytotoxic immune cell (e.g., NK cell, NK-92 cell and its derivative, NKT cell, genetically engineered NKT cells, etc.) so that the NK and/or NKT cells initiate antibody-dependent cell-mediated cytotoxicity (ADCC) against the mesenchymal tumor cell by binding the Fc portion of the binding molecule (in the protein complex) via CD16. Therefore, the inventors contemplate that in some embodiments, the binding molecules can be administered to the patient (e.g., either systemically or locally by intravenous injection or intratumoral injection) at least 6 hours, at least 12 hours, at least 24 hours, at least 3 days, at least 7 days before administering cytotoxic immune cell to the patient.

As used herein, the term “administering” binding molecules and/or cytotoxic immune cell refers to both direct and indirect administration of the binding molecules and/or cytotoxic immune cell, wherein direct administration of binding molecules and/or cytotoxic immune cell is typically performed by a health care professional (e.g., physician, nurse, etc.), and wherein indirect administration includes a step of providing or making available binding molecules and/or cytotoxic immune cell to the health care professional for direct administration (e.g., via injection, etc.).

Yet, the inventors also contemplate that the order and manner of administering the binding molecules and/or cytotoxic immune cell may vary depending on type of binding molecules, type of molecular markers, type of cytotoxic immune cells, health status of the patient, previous history of cancer treatment, and so on. Thus, in some embodiments, the binding molecules and/or cytotoxic immune cell can be administered to the patient substantially simultaneously (e.g., within 5 min, within 10 min, within 1 hour, within 2 hours, etc.). In such embodiments, it is preferred that the binding molecules and the cytotoxic immune cell are administered using the same administrating method (e.g., intratumoral injection) such that both binding molecules and cytotoxic immune cell can contact the tumor and infiltrate into the tumor almost simultaneously.

Also, it is contemplated that the dose and schedule of administering binding molecules and/or cytotoxic immune cell may vary depending on type of binding molecules, type of molecular markers, type of cytotoxic immune cells, health status of the patient, previous history of cancer treatment, and so on. Most typically, the antibody will be administered in dosages between 0.01 mg/kg and 150 mg/kg, or between 0.01 mg/kg and 15 mg/kg, or between 0.1 mg/kg and 5 mg/kg, or between 1mg/kg and 10 mg/kg, for example, by weekly intravenous injection over 1-2 hours. Similarl y, Similarly, NK cells or NKT cells (either naïve or genetically engineered) may be transfused over several administrations, for example weekly, typically in an amount of between 10⁴ cells/kg and 10¹⁰ cells/kg, or between 10⁵ cells/kg and 10⁹ cells/kg, or between 10⁶ cells/kg and 10⁸ cells/kg per transfusion.

Alternatively, it is also contemplate that the cytotoxic immune cell is contacted with binding molecules such that binding molecules can form a CD16-binding molecule complex on the cytotoxic immune cell surface. In such embodiment, it is preferred that the contact between binding molecule and the cytotoxic immune cell is completed close to the administration of the cytotoxic immune cell (with binding molecule) to the tumor (e.g., less than 1 hour, less than 30 min, less than 10 min before administering the cytotoxic immune cell to the patient, etc.).

In additionally contemplated aspects, it should be appreciated that such treatment may be further supplemented by administration of one or more drugs or modalities that inhibit immune suppression and/or that stimulate an immune response. For example, immune response may be further stimulated by administering to the cancer stem cell or a tumor microenvironment an immune stimulating cytokine, including IL-2, IL-12, IL-15, IL-15 superagonist (e.g., ALT803), and/or an IL-15 superagonist hybrid comprising a chemokine or chemokine portion such as CXCL14. Of course, it should be noted that such administration may be performed in a conventional manner, or via expression of the cytokine(s) in the NK cell. Similarly, it should be appreciated that immune stimulation may be performed using one or more chemokines (and especially pro-inflammatory chemokines) that will attract T cells and/or NK cells. For example, suitable chemokines include CCL2, CCL3 and CCL5, CXCL1, CXCL2, CXCL8, and CXCL14.

Moreover, and especially where calreticulin is targeted by a-calreticulin antibody, it is contemplated that the immune response by NK cells may be further enhanced by administering to the cancer stem cell or tumor microenvironment a CD47 antagonist or a SHPS-1 antagonist, which reduces down-regulation of calreticulin-mediated cytotoxicity. Alternatively, or additionally, one or more agents may be administered to the cancer stem cell or tumor microenvironment that up-regulates surface expression of calreticulin. For example, various anthracyclines or thapsigargin are known to increase surface expression of calreticulin. Similarly, a radiosensitizing drug may be administered to the cancer cell or cancer stem cell to so increase cell stress such that some stress-induced cell surface protein, especially NK cell receptor ligand (e.g., NKG2D ligand, etc.) can be upregulated on the cell surface of the cancer cell or cancer stem cell. Advantageously, such drug may be coupled to nanoparticulate albumin (e g , albumin-coupled paclitaxel) such that the drug can be readily infiltrated into the tumor microenvironment and gain an access to the mesenchymal tumor cells.

Where desired, additional oxygen may be provided to the tumor microenvironment (e.g., via oxygen hyperbaric treatment, etc.) to so reduce the otherwise immunosuppressive environment. Alternatively, or additionally, at least some of the immunosuppressive environment is produced by the tumor via TGF-β secretion, leading to attraction/activation of myeloid derived suppressor cells (MDSC). Thus, treatment with antibodies against TGF-β or agents that disrupt TGF-β signaling may also be employed. Similarly, tumor stem cells are also known to secrete IL-8 in an autocrine loop to develop and maintain EMT/mesenchymal state. To counteract such immune evasion, the inventors contemplate that IL-8 signaling can be blocked using an IL-8 antibody or any other binding molecules to IL-8 (e.g., scFv fragment, etc.), and it should be noted that such antibodies are well known in the art (see e.g., J. Immunol. Methods 1992 149:227 or WO 1997/001354). Alternatively, or additionally, IL-8 signaling may also be performed using non-antibody binders to IL-8 (e.g., as prepared by RNA display), and RNAi that reduces or abrogates IL-8 expression. In still further aspects of blocking IL-8 signaling, it is contemplated that various IL-8 antagonist may be administered, and especially contemplated IL-8 antagonists include various 2-amino-3-heteroaryl-quinoxalines (see e.g., Bioorg Med Chem. 2003 Aug. 15; 11(17):3777-90).

Also, any other MDSC inhibitors including MDSC recruitment inhibitor, MDSC expansion inhibitor, MDSC differentiation inhibitor, and/or MDSC activity inhibitor are contemplated. For example, MDSC recruitment inhibitor may include one or more antagonists of one or more colony-stimulating factor 1 receptor (CSF-R), granulocyte colony-stimulating factor (G-CSF), C-C motif chemokine ligand 2 (CCL2), or C-X-C chemokine receptor type 4 (CXCR4). The antagonist may include small molecule inhibitors, antibodies or fragments thereof that bind to the target molecule, single-chain variable fragment (scFv) molecule binding to the target molecule, or any other suitable binding molecules. For example, the antagonist of CSF-R may include a small molecule inhibitor (e.g., Pexidartinib, etc.) or one or more monoclonal antibodies against CSF-R (e.g., Emactuzumab, AMG820, imc-CS4, MCS110, etc.). Alternatively or additionally, expansion of the MDSCs in the tumor may be inhibited by administering gemcitabine, amino bisphosphonates, sunitinib, or celecoxib, and differentiation of MDSCs in the tumor may be inhibited by taxanes, curcumin, or Vitamin D3. In addition, MDSC activity in the tumor may be inhibited by administration of amiloride, CpG, COX2 inhibitors, PDE-5 inhibitors, or PGE2 inhibitors.

Additionally, or alternatively, the agent may also be a CXCR1 inhibitor and/or a CXCR2 inhibitor. There are various such inhibitors known in the art, and appropriate inhibitors various 2-amino-3-heteroaryl-quinoxalines (see e.g., Bioorg Med Chem. 2003 Aug. 15; 11(17):3777-90), 6-Chloro-3-[[[(2,3-dichlorophenyl)amino]carbonyl]amino]-2-hydroxybenzenesuffonamide (SB332235), or N-(2-Bromophenyl)-N′-(7-cyano-1H-benzotriazol-4-yl)urea (SB265610). If inhibitors with higher specificity are desired, SCH-527123 and SCH-479833 may be employed that will selectively inhibit CXCR2 and CXCR1, respectively (see e.g., Clin Cancer Res. 2009 Apr. 1; 15(7):2380-6). Of course, it should be appreciated that the CXCR1/2 pathway activity may also be inhibited by one or more agents that interfere with the elements of the signaling chain. In still another example, the activation of the IL-8 receptor, including CXCR1/2, can be inhibited using reparixin (also known as repertaxin, see e.g., Biol Pharm Bull. 2011; 34(1):120-7), or the IL-8-mediated signaling cascade through CXCR1/2 can be inhibited by blocking one or more elements in the signaling pathways. Thus, inhibitors can also target CXCR1 and 2 signaling pathways by targeting PI3kinase, pAkt, or mTOR for CXCR1 signaling inhibition, and/or RhoGTPase, RacGTPas, and Ras, Raf, Mek, or pErk for CXCR2 signaling inhibition. Since IL-8 signaling also at least indirectly affects MDSCs, it is expected that at least some of the above agents will reduce activity or recruitment of MDSC to the tumor environment.

Additionally, the inventors further contemplate administering another reagent that inhibit EMT of the tumor cell or reverse the EMT process of the tumor cell, or even promote mesenchymal to epithelial transition (MET) of the tumor cell. For example, during the EMT process, TGF-β induces isoform switching of FGF Receptor 2 (e.g., from isotype IIIb to IIIc), and it is contemplated that inhibiting TGF-β activity in the tumor cells (e.g., using dominant negative form of TGF-β RII, monoclonal antibodies against TGF-beta 1 and beta 2, including lerdelimumab and metelimumab, etc.) may reduce or prohibit the isoform switching of FGF Receptor 2 to so prevent EMT of the tumor cell. In another example, MET may be induced in vitro by administering 8-bromo-cAMP, Taxol, or Adenosine 3prime,5prime-cyclic Monophosphate, N6-Benzoyl-Sodium Salt, which activate protein kinase A (PKA). MET of the tumor cell can be also induced by administering a recombinant virus encoding recombinant E-Cadherin or regulatory RNA inhibiting N-Cadherin expression to stimulate of E-Cadherin overexpression and reduce N-Cadherin expression. Further, MET of the tumor cell can be also induced by EGFR inhibition and/or down-regulation of Snail, Slug, Zeb-1, Zeb-2, and/or N-cadherin (e.g., using siRNA, miRNA, shRNA, or other regulatory small molecule reducing the post-transcriptional expression, etc.).

In still further contemplated aspects, other inhibitory reagents to immune suppressive cells may be administered concurrently with the binding molecule and/or cytotoxic immune cells or before administering the binding molecule and/or cytotoxic immune cells. Especially contemplated reagents include RP-182 (U.S. Pat. No. 9,492,499) to inhibit or kill M2 macrophages, gemcitabine, cis-platinum, and/or cyclophosphamide to reduce or inhibit regulatory T cells (Tregs).

The dose and schedule of administering of additional reagents, including MDSC inhibitors, CXCR1 inhibitor and/or a CXCR2 inhibitor or other inhibitory reagents, may vary depending on the type of reagents and dose and schedule of administering binding molecule and/or cytotoxic immune cells. For example, with respect to MDSC inhibitor(s), it is contemplated that MDSC inhibitor(s) can be administered at least 1 day, 3 days, 5 days, 7 days before administering binding molecule and/or cytotoxic immune cells to change the tumor microenvironment more amenable to the immune therapy. However, it is also contemplated that the MDSC inhibitor(s) can be administered almost simultaneously or even after administering binding molecule and/or cytotoxic immune cells (e.g., in 3 hours, in 6 hours, in 12 hours, in 1 day, in 3 days, etc.).

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1-100. (canceled)

101. A method of targeting a cancer stem cell, comprising:
providing an antibody having binding specificity towards an antigen that is specific to a mesenchymal state of a tumor stem cell;
providing a genetically modified natural killer (NK) cell that expresses a CD16 receptor and that has granulysin and granzyme mediated cytotoxic activity under hypoxic conditions; and
contacting the cancer stem cell with the antibody and the genetically modified NK cell to allow antibody-mediated binding of the genetically modified NK cell to the cancer stem cell.

102. The method of claim 101 wherein the antigen is at least one of calreticulin, PD-L1, and c-MET.

103. The method of claim 101 wherein the antibody is a bi-specific antibody having binding specificity against at least two of calreticulin, PD-L1, and c-MET.

104. The method of claim 101 wherein the genetically modified NK cell is genetically modified to express at least one of a high affinity variant CD16 and endoplasmic restricted IL-2.

105. The method of claim 101 wherein the step of contacting the cancer stem cell with the antibody and the genetically modified NK cell is performed while the cancer stem cell is within a tumor microenvironment.

106. The method of claim 101 wherein the step of contacting the cancer stem cell with the antibody and the genetically modified NK cell is performed while the cancer stem cell is under hypoxic conditions.

107. The method of claim 101 further comprising a step of administering to the cancer stem cell or a tumor microenvironment an immune stimulating cytokine.

108. The method of claim 101 further comprising a step of administering to the cancer stem cell or a tumor microenvironment an IL-15, an IL-15 superagonist, and/or an IL-15 superagonist hybrid comprising a chemokine or chemokine portion.

109. The method of claim 101 further comprising a step of administering to the cancer stem cell or tumor microenvironment an agent that down-regulates suppressor cells, optionally gemcitabine, cis-platinum, or cyclophosphamide.

110. The method of claim 101 further comprising a step of administering to the cancer stem cell or a tumor microenvironment a peptide that down-regulates or kills M2 macrophages, optionally a RP-182 or an antibody against B7-H4.

111. The method of claim 101 further comprising a step of administering to the cancer stem cell or a tumor microenvironment an IL-8 antibody, an IL-8 antagonist, a CXCR1 inhibitor, and/or a CXCR2 inhibitor.

112. A method of treating a cancer, comprising:
co-administering to a patient having the cancer an antibody and a genetically modified natural killer (NK) cell;
wherein the antibody has binding specificity towards an antigen that is specific to a mesenchymal state of a tumor stem cell;
wherein the genetically modified NK cell expresses a CD16 receptor and has granulysin and granzyme mediated cytotoxic activity under hypoxic conditions; and
wherein the antibody and the genetically modified NK cell are administered to the patient to allow antibody-mediated binding of the genetically modified NK cell to the cancer stem cell in a tumor microenvironment.

113. The method of claim 112 wherein the antigen is at least one of calreticulin, PD-L1, and c-MET.

114. The method of any one of claims 112 further comprising a step of administering to the patient or tumor microenvironment an IL-15, an IL-15 superagonist, and/or an IL-15 superagonist hybrid comprising a chemokine or chemokine portion.

115. The method of any one of claims 112 further comprising a step of administering to the patient or tumor microenvironment a peptide that down-regulates or kills M2 macrophages, optionally a RP-182 or an antibody against B7-H4.

116. The method of any one of claims 112 further comprising a step of administering to the patient or tumor microenvironment an IL-8 antibody, an IL-8 antagonist, a CXCR1 inhibitor, and/or a CXCR2 inhibitor.

117. A method of targeting a cancer cell in a hypoxic environment in which the cancer cell has reduced cell division and/or activity in an apoptotic pathway, comprising:
identifying the cancer cell as expressing an antigen that is specific to a mesenchymal state of a tumor cell;
providing an antibody having binding specificity towards the antigen;
providing a genetically modified natural killer (NK) cell that expresses a CD16 receptor and that has granulysin and granzyme mediated cytotoxic activity under hypoxic conditions; and
contacting the cancer stem cell with the antibody and the genetically modified NK cell to allow antibody-mediated binding of the genetically modified NK cell to the cancer stem cell.

118. The method of claim 117 wherein the antigen is at least one of calreticulin, PD-L1, and c-MET.

119. The method of any one of claims 117 wherein the step of contacting the cancer stem cell with the antibody and the genetically modified NK cell is performed while the cancer stem cell is within a tumor microenvironment.

120. The method of any one of claims 117 wherein the step of contacting the cancer stem cell with the antibody and the genetically modified NK cell is performed while the cancer stem cell is under hypoxic conditions.
